(19) United States
(12) Patent Application Publication
(10) Pub. No.: US 2017/0231898 A1
(43) Pub. Date: Aug. 17, 2017

(54) AGENTS FOR THE TEMPORARY SHAPING OF KERATIN-CONTAINING FIBERS, INCLUDING A HYDROPHOBICALLY MODIFIED METAL OXIDE POWDER, AN AMINOFUNCTIONAL SILICONE AND WATER

(71) Applicant: Henkel AG & Co. KGaA, Duesseldorf (DE)

(72) Inventors: Thorsten Knappe, Schenefeld (DE); Ulrike Heinsohn, Hamburg (DE)

(73) Assignee: Henkel AG & Co. KGaA, Duesseldorf (DE)

(21) Appl. No.: 15/586,576

(22) Filed: May 4, 2017

Related U.S. Application Data
(63) Continuation of application No. PCT/EP2015/072835, filed on Oct. 2, 2015.

(30) Foreign Application Priority Data
Nov. 7, 2014 (DE) 102014222801.8

(51) Int. Cl. A61K 8/898; B65D 83/14; A45D 7/04; A45D 34/04; A61Q 5/06; A61K 8/25

(52) U.S. Cl. CPC A61K 8/898; A61Q 5/06; A61K 8/25; A45D 7/04; A45D 34/04; B65D 83/752; A61K 2800/87

(57) ABSTRACT

Cosmetic agents for temporarily shaping keratin fibers, comprising a) a cosmetic preparation, including, based on the total weight of the cosmetic preparation, a1) 0.01 to 1.0 wt.-% of at least one hydrophobically modified metal oxide powder; a2) 0.01 to 0.5 wt.-% of at least one aminofunctional silicone; and a3) 40 to 95 wt.-% of water, are characterized by a high level of hold and volume.

### FIELD OF THE INVENTION

The present invention generally relates the specialist technical field of temporary re-shaping of keratin-containing fibers, in particular human hair. The subject of the invention is constituted by cosmetic agents including at least one hydrophobized metal oxide powder, aminofunctional silicone, and water, and also by cosmetic products comprising a dispensing device having a spray valve, and also by the aforementioned cosmetic agents. Further subjects of the present invention are the use of these cosmetic agents and products for the temporary re-shaping of keratin-containing fibers and also corresponding application methods.

### BACKGROUND OF THE INVENTION

A nice-looking hairstyle is nowadays generally regarded as an essential part of a well-groomed appearance. Time and time again, based on current fashion trends, hairstyles that can be constructed with many types of hair only using firming active ingredients or that stay in place for a relatively long period of time up to several days are considered chic. Hair treatment agents that permanently or temporarily shape the hair therefore play an important role. Whereas, in the case of permanent re-shaping, the chemical structure of the keratin-containing fibers is modified by reduction and oxidation, no such modification of the chemical structure takes place in the case of temporary re-shaping. Corresponding agents for temporary shaping usually contain synthetic polymers and/or waxes as firming active ingredient.

The most important property of an agent for temporarily shaping keratin fibers, also referred to hereinafter as styling agents, lies in providing the treated fibers with the greatest possible hold in the newly modeled form—i.e. a form impressed on the fibers. If the keratin fibers are human hair, reference is also made to a strong hairstyle hold or to a high holding power of the styling agent. The hold of a hairstyle is determined fundamentally by the type and quantity of the used firming active ingredients, however the further constituents of the styling agent and also the application form can also have an effect.

In addition to a high holding power, styling agents should also satisfy a wide range of further requirements. These can be divided roughly into properties on the hair, properties of the respective formulation, for example properties of the sprayed aerosol or non-aerosol, and properties that concern the handling of the styling agent, wherein the properties on the hair are attributed particular importance. In particular, moisture resistance, low stickiness, and a balanced conditioning effect can be cited. A further key point is the building of hair volume and hair fullness. Furthermore, a styling agent should be universally usable for all hair types where possible and should be mild on the hair and skin.

In order to satisfy the different requirements, a multiplicity of synthetic polymers which are used in styling agents have been developed in the prior art as firming active ingredients. These polymers can be divided into cationic, anionic, non-ionic and amphoteric firming polymers. When applied to the hair, the polymers ideally provide a polymer film, which on the one hand gives the hairstyle a stronger hold, but on the other hand is sufficiently flexible so as not to break under load. If the polymer film is too brittle, this results in the formation of what are known as film flakes or residues, which come loose as the hair moves and give the impression that the user of the corresponding styling agent has dandruff. Similar problems are encountered when wax is used as a firming active ingredient in the styling agent.

Agents for assisting the temporary re-shaping of keratin-containing fibers can be produced for example as a hairspray, hair wax, hair gel, or hair mousse. In particular, the application in the form of a spray by means of a spraying device is highly popular. However, the application by means of a spraying device requires additional measures, depending on the nature of the styling agent. By way of example, an application by spraying of thickened hair gels in the prior art was previously possible only with use of a specific combination of thickening agent and styling polymer, since only in this way could a sufficient nebulization of the hair gel be ensured by means of the spraying application.

The combination of a carbomer and PVP/VA styling polymers used in the prior art can indeed provide sufficient nebulization, but does not provide satisfactory prolonged hold or volume effect and therefore fails to satisfy consumer needs.

The object of the present invention was therefore to provide agents for temporarily shaping keratin-containing fibers, which agents can be applied in an effective manner as a targeted spray mist to the keratin-containing fibers. The agents for temporarily shaping keratin-containing fibers should also have a high holding power, in particular prolonged holding power, and a high volume effect.

### BRIEF SUMMARY OF THE INVENTION

A first subject of the present invention is thus a cosmetic agent for temporarily shaping keratin fibers, comprising

a) a cosmetic preparation including, based on the total weight of the cosmetic preparation,

a1) 0.01 to 1.0 wt.-% of at least one hydrophobically modified metal oxide powder;

a2) 0.01 to 0.5 wt.-% of aminofunctional silicone;

a3) 40 to 95 wt.-% of water.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It has now surprisingly been found that an aqueous preparation of a specific combination of hydrophobically modified metal oxide powder with aminofunctional silicones is suitable for solving the above-described problem. This specific combination is characterized not only by a high long-term hold and a good volume effect, but in addition can be nebulized in an excellent manner and therefore is also suitable for application by spraying. The hairstyles treated by means of cosmetic agents according to the invention can be easily combed, have a pleasant feel, and in addition have high hair volume and high hair fullness.

The agents according to the invention contain a hydrophobically modified metal oxide powder as a first essential constituent. With regard to their cosmetic effect, producibility and applicability, preferred agents are characterized in that the proportion by weight of the hydrophobically modified metal oxide powder a1) in the total weight of the cosmetic preparation a) is 0.02 to 1.0 wt.-%, preferably 0.03 to 0.8 wt.-%, and in particular 0.05 to 0.2 wt.-%.

In the sense of the invention, “hydrophobically modified” or “hydrophobized” are understood to mean metal oxides which have been modified at least at the surface of the particles in such a way that the modified particle is wetted to a lesser extent by water than the non-modified particle. In particular, silanized, hydrophobized metal oxides are preferred. At least one representative of the group formed from silanes, halosilanes, alkoxysilanes, and silazanes is preferably suitable in accordance with the invention as reagent for silanizing the metal oxide. Preferably suitable hydrophobized metal oxides of the hydrophobized metal oxide powder are selected in accordance with the invention from at least one representative of the group formed from hydrophobized silicates, hydrophobized aluminum silicates, hydrophobized titanium dioxide, and hydrophobized silicon dioxide. Hydrophobized silicates have proven to be particularly suitable for the production of the cosmetic agents according to the invention, wherein fumed silicas post-treated by silanization or by reaction with polydimethyl siloxane have particular advantages.

The agents according to the invention preferably contain hydrophobized silicon dioxide. The type of hydrophobically modified silicon dioxide a1) is not limited in principle, however at least one silanized, hydrophobized silicon dioxide is preferably used as hydrophobized metal oxide powder on account of the cosmetic properties of corresponding agents.

At least one representative of the group formed from silanes, halosilanes, alkoxysilanes, and silazanes is preferably suitable in accordance with the invention as reagent for silanization of the silicon dioxide.

Preferred representatives of the group of silanes are hexa(C₁-C₂₀)alkyldisilanes, in particular hexamethyldisilane.

If a halosilane is used as silylation agent, at least one compound from the group formed from the compounds

[(C₁-C₂₀)alkyl]_{z′}SiX_{(4-z′)}

X₃Si[(CH₂)—R]

X₂[(C₁-C₂₀)alkyl]Si(CH₂)ₙ—R

[(C₁-C₂₀)alkyl](_{y+1})[R—(CH₂)ₙ]_{(2-y′)}SiX

in which
X means a chlorine, bromine or iodine atom,
z′ is a number 1, 2 or 3,
y′ is a number 0, 1 or 2,
n is an integer from 1 to 20, and
R stands for a group from
(C₁-C₁₀)alkyl-, aryl-, (C₁-C₆)perfluoroalkyl-, —NH₂, —N₃, —SCN, —CH═CH₂, —O(O)C—C(CH₃)═CH₂, —OCH₂—CH═CH₂,

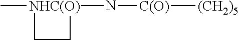

—NH—C(O)O-Me, —NH—C(O)O-Et, —NH—(CH₂)₃—Si(O(C₁-C₆)alkyl)₃
is selected as preferred halosilane.

If an alkoxysilane is used as silylation agent, at least one compound from the group formed from the compounds

[(C₁-C₂₀)alkylO]_{z}Si(C₁-C₂₀)alkyl_{(4-z)}

[(C₁-C₂₀)alkylO]_{z}Si[(CH₂)ₙ—R]_{(4-z)}

[(C₁-C₂₀)alkylO]₂[(C₁-C₂₀)alkyl]Si(CH₂)ₙ—R

[(C₁-C₂)alkylO][(C₁-C₂₀)alkyl]₂Si(CH₂)ₙ—R

[(C₁-C₂₀)alkylO][(C₁-C₂₀)alkyl]Si(CH₂)ₙ—R]₂

(C₁-C₂₀alkyl)₃SiO—C(CH₃)═N—Si(C₁-C₂₀)alkyl₃,

in which
n is an integer from 1 to 20, and
z means a number 1, 2 or 3,
R stands for a group from
(C₁-C₂₀)alkyl-, aryl-, (C₁-C₆)perfluoroalkyl-, —NH₂, —N₃, —SCN, —CH═CH₂, —O(O)C—C(CH₃)═CH₂, —OCH₂—CH═CH₂,

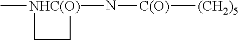

—NH—C(O)O-Me, —NH—C(O)O-Et, —NH—(CH₂)₃—Si(O(C₁-C₆)alkyl)₃
is selected as preferred alkoxysilane.

At least one compound from the class of disilazanes, in particular at least one compound from disilazanes of formula

R′₂R″Si—NH—SiR′₂R″

in which
R′ means a (C₁-C₂₀)alkyl group and
R″ means a (C₁-C₂₀)alkyl group or a vinyl group, is selected as preferred silazane. A particularly preferred silazane is hexamethyldisilazane.

All of the above-mentioned alkyl groups, whether (C₁-C₆)alkyl, (C₁-C₁₀)alkyl or (C₁-C₂₀)alkyl, can be cyclic and also linear or branched. Examples of alkyl groups that can be used in accordance with the invention are methyl, ethyl, n-propyl, isopropyl, n-butyl, cyclopentyl, cyclohexyl, n-decyl, lauryl, myristyl, cetyl, stearyl, isostearyl and behenyl.

An example of an aryl group according to the invention is the phenyl group.

Examples of a (C₁-C₆)perfluoroalkyl group according to the invention are trifluoromethyl, perfluoromethyl, perfluoropropyl, and perfluorohexyl.

Hydrophobized silicon dioxides which are obtained by silanization of pyrogenic silicon dioxide are preferably used.

Silanized, hydrophobized silicon dioxides are preferably selected in particular from at least one compound of the group that is formed from trimethylsilylate-coated silicon dioxide, dimethylsilylate-coated silicon dioxide, octylsilylate-coated silicon dioxide.

Preferred cosmetic agents are characterized in that the hydrophobically modified metal oxide powder a1) is selected from the group of hydrophobized silicates obtained by silanization of pyrogenic silicon dioxide.

A multiplicity of suitably hydrophobically modified silicon dioxides is commercially obtainable. The following can be cited by way of example, Aerosil® R104 V, Aerosil® R106, Aerosil® R202, Aerosil® R805, Aerosil® R812, Aerosil® R812S, Aerosil® R972 and Aerosil® R8200, all Degussa products, and HDK® H2000, HDK®H2050 and HDK® H3004, all Wacker products. The silicon dioxide with the INCI name Silica Dimethyl Silylate is very particularly preferably used and is sold by the company Degussa under the name Aerosil® R972. To summarize, cosmetic agents are preferred in which the hydrophobically modified metal oxide powder a1) is selected from the compounds having the INCI names Silica Dimethyl Silylate (for example Aerosil R792, Aerosil R794), Silica Dimethicone Silylate (for example Aerosil R202), and Silica Silylate (for example Aerosil R805, Aerosil R812, Aerosil R816). Compounds with the INCI name Silica Dimethyl Silylate are particularly preferred.

The hydrophobically modified silicon dioxides with the INCI name Silica Dimethyl Silylate enable a simplified production of cosmetic agents according to the invention, which are characterized additionally by an improved applicability and cosmetic effect, compared to the other previously described hydrophobically modified silicon dioxides. The production of the particularly preferred hydrophobized silicon dioxides with the INCI name Silica Dimethyl Silylate can be implemented for example by reacting fumed silica with dimethyldichlorosilane. A particularly preferred subject of the present invention is therefore a cosmetic agent which includes a hydrophobically modified metal oxide powder obtained by reacting fumed silica with dimethyldichlorosilane.

The particle diameter of the primary particles of preferred hydrophobically modified metal oxides a1), in particular the hydrophobically modified metal oxides with the INCI name Silica Dimethyl Silylate, is preferably less than 5 μm, particularly preferably less than 1 μm, and in particular between 1 and 50 nm.

Hydrophobically modified metal oxides a1), in particular hydrophobically modified metal oxides with the INCI name Silica Dimethyl Silylate, which have a specific BET surface area between 10 and 400 m²/g, preferably between 40 and 300 m²/g, and in particular 80 to 150 m²/g, are also preferred.

The agents according to the invention contain, as second essential constituent, the aminofunctional silicone a2). Corresponding silicones which have at least one amino group, which may or may not be substituted, are referred to in accordance with the INCI declaration as amodimethicones.

For the application and the cosmetic effect of the cosmetic agents, it has proven to be advantageous if the proportion by weight of the aminofunctional silicone a2) in the total weight of the cosmetic preparation a) is 0.02 to 0.4% by weight, preferably 0.03 to 0.3% by weight, and in particular 0.05 to 0.5% by weight.

Preferred agents according to the invention contain one or more aminofunctional silicones of formula M(RₐQ_{b}SiO_{(4-a-b)/2})ₓ(R_{c}SiO_{(4-c)/2})_{y}M (Si-1), wherein, in the above formula

R is a hydrocarbon or a hydrocarbon group having 1 to approximately 6 carbon atoms, preferably methyl,
Q is a polar group of general formula —R¹HZ, preferably a group of formula —CH₂CH₂CH₂NHCH₂CH₂NH₂,
R¹ is a bivalent, linking group, which is bound to hydrogen and the group Z, composed of carbon and hydrogen atoms, carbon, hydrogen and oxygen atoms, or carbon, hydrogen and nitrogen atoms, and
Z is an organic, aminofunctional group, which includes at least one aminofunctional grouping, wherein it is preferably a —NHCH₂CH₂NH₂ group or a group of formula —CH₂CH₂CH₂NHCH₂CH₂NH₂,
a assumes values in the range of from approximately 0 to approximately 2,
b assumes values in the range of from approximately 1 to approximately 3,
a+b is less than or equal to 3, and
c is a number in the range of from approximately 1 to approximately 3, and
x is a number in the range of from 1 to approximately 2,000, preferably from approximately 3 to approximately 50, and most preferably from approximately 3 to approximately 25, and
y is a number in the range of from approximately 20 to approximately 10,000, preferably from approximately 125 to approximately 10,000, and most preferably from approximately 150 to approximately 1,000, and
M is a suitable silicone terminal group, as is known in the prior art, preferably trimethylsiloxy.

Examples of R¹ include methylene, ethylene, propylene, hexamethylene, decamethylene —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Preferred cosmetic or dermatological preparations according to the invention contain an aminofunctional silicone of formula R′ₐG₃₋ₐ-Si(OSiG₂)ₙ-(OSiG_{b}R′_{2-b})ₘ—O-SiG₃₋ₐ-R′ₐ (Si-2), in which:

G is —H, a phenyl group, —OH, —O—CH₃, —CH₃, —O—CH₂CH₃, —CH₂CH₃, —O—CH₂CH₂CH₃, —CH₂CH₂CH₃, —O—CH(CH₃)₂, —CH(CH₃)₂, —O—CH₂CH₂CH₂CH₃, —CH₂CH₂CH₂CH₃, —O—CH₂CH(CH₃)₂, —CH₂CH(CH₃)₂, —O—CH(CH₃)CH₂CH₃, —CH(CH₃)CH₂CH₃, —O—C(CH₃)₃, —C(CH₃)₃;
a stands for a number between 0 and 3, in particular 0;
b stands for a number between 0 and 1, in particular 1;
m and n are numbers of which the sum (m+n) is between 1 and 2000, preferably between 50 and 150, wherein n preferably assumes values from 0 to 1999 and in particular from 49 to 149, and m preferably assumes values from 1 to 2000, in particular from 1 to 10,
R′ is a monovalent group selected from

-Q-N(R″)—CH₂—CH₂—N(R″)₂

-Q-N(R″)₂

-Q-N⁺(R″)₃A⁻

-Q-N⁺H(R″)₂A⁻

-Q-N⁺H₂(R″)A⁻

-Q-N(R″)—CH₂—CH₂—N⁺R″H₂A⁻,

wherein each Q stands for a chemical bond, —CH₂—, —CH₂—CH₂—, —CH₂CH₂CH₂—, —C(CH₃)₂—, CH₂CH₂CH₂CH₂—, —CH₂C(CH₃)₂—, —CH(CH₃)CH₂CH₂—,
R″ stands for identical or different groups from the group —H, -phenyl, -benzyl, —CH₂—CH(CH₃)Ph, the C₁₋₂₀ alkyl groups, preferably —CH₃, —CH₂CH₃, —CH₂CH₂CH₃, —CH(CH₃)₂, —CH₂CH₂CH₂CH₃, —CH₂CH(CH₃)₂, —CH(CH₃)CH₂CH₃, —C(CH₃)₃, and A represents an anion which is preferably selected from chloride, bromide, iodide and methosulfate.

Particularly preferred agents according to the invention are characterized in that they contain at least one aminofunctional silicone of formula (Si2-a)

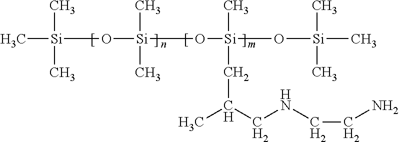

in which m and n are numbers of which the sum (m+n) is between 1 and 2000, preferably between 50 and 150, wherein n preferably assumes values from 0 to 1999 and in particular from 49 to 149, and m preferably assumes values from 1 to 2000, in particular from 1 to 10.

In a further preferred embodiment, cosmetic agents according to the invention contain an aminofunctional silicone of formula (Si-2b)

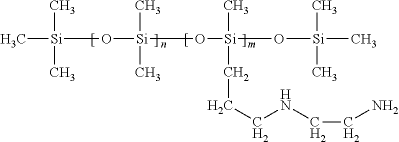

in which m and n are numbers of which the sum (m+n) is between 1 and 2000, preferably between 50 and 150, wherein n preferably assumes values from 0 to 1999 and in particular from 49 to 149, and m preferably assumes values from 1 to 2000, in particular from 1 to 10.

These silicones of formula (Si2-a) and (Si2-b) are referred to as amodimethicones in accordance with the INCI declaration. Corresponding amodimethicones are obtainable for example in the form of an emulsion as commercial product Dow Corning 939 or as commercial product Dow Corning 949.

In an alternative embodiment, the aminofunctional silicone a2), however, is a functionalized amodimethicone. Corresponding functionalized amodimethicones are described for example by the formula (Si-2c)

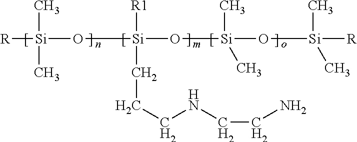

in which
R stands for —OH, a (C₁ to C₂₀) alkoxy group (which may or may not be ethoxylated and/or propoxylated) or a —CH₃ group,
R1 stands for —OH, a (C₁ to C₂₀) alkoxy group or a —CH₃ group, and m, n1 and n2 are numbers of which the sum (m+n1+n2) is between 1 and 2000, preferably between 50 and 150, wherein the sum (n1+n2) preferably assumes values from 0 to 1999 and in particular from 49 to 149, and m preferably assumes values from 1 to 2000, in particular from 1 to 10.

By way of example, aminofunctional silicones a2) from the group of hydroxylamino-modified silicones, in which in the above formula (Si-2b) the group R stands for —OH, are preferred. Corresponding silicones with the INCI name Amodimethicone are obtainable for example as commercial product Wacker Belsil ADM 652.

If the group R stands for a (C₁ to C₂₀) alkoxy group (which may or may not be ethoxylated and/or propoxylated), for example bis(C₁₃₋₁₅ alkoxy) PG amodimethicone (obtainable for example as commercial product: DC 8500 from the company Dow Corning) and trideceth-9 PG amodimethicone (obtainable for example as commercial product Silcare Silicone SEA from the company Clariant) fall under this general formula (Si-2b).

Regardless of which aminofunctional silicones are used, cosmetic preparations according to the invention which contain an aminofunctional silicone of which the amine value lies above 0.25 meq/g, preferably above 0.3 meq/g, and in particular above 0.4 meq/g, are preferred. The amine value stands here for the milli-equivalents of amine per gram of the aminofunctional silicone. It can be determined by titration and can also be specified in the unit mg KOH/g.

As third essential constituent, cosmetic agents according to the invention contain 40 to 95% by weight of water, wherein, with regard to the production and application of the cosmetic agents, it is preferred that the proportion by weight of the water a3) in the total weight of the cosmetic preparation a) is 60 to 95% by weight, preferably 70 to 95% by weight, and in particular 80 to 95% by weight.

Agents according to the invention are present in the form of flowable suspensions.

Besides the above-described contents, the cosmetic agents according to the invention can contain further active ingredients, auxiliaries and nourishing ingredients.

A first group of preferred further active ingredients is formed by the aminofunctional silicone a2) of various quaternary ammonium compounds a4), preferably the quaternary ammonium compound from the group of trimethylalkylammonium halides.

Corresponding substances are a preferred constituent of the cosmetic preparations a).

In particular, the compounds of formula (Tkat1-1) belong to the group of trimethylalkylammonium halides.

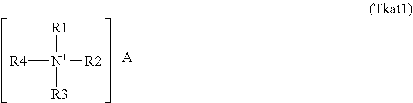

In the formula (Tkat1), R1, R2, R3 and R4 each stand, independently of one another, for hydrogen, a methyl group, a phenyl group, a benzyl group, for a saturated, branched or unbranched alkyl group having a chain length of from 8 to 30 carbon atoms, which can be substituted with one or more hydroxyl groups as appropriate. A stands for a physiologically acceptable anion, for example halides such as chloride or bromide, and methosulfates.

Examples of compounds of formula (Tkat1) are lauryl trimethyl ammonium chloride, cetyl trimethyl ammonium chloride, cetyl trimethyl ammonium bromide, cetyl trimethyl ammonium methosulfate, dicetyl dimethyl ammonium chloride, tricetyl methyl ammonium chloride, stearyl trimethyl ammonium chloride, distearyl dimethyl ammonium chloride, lauryl dimethyl benzyl ammonium chloride, behenyl trimethyl ammonium chloride, behenyl trimethyl ammonium bromide, behenyl trimethyl ammonium methosulfate. A particularly preferred quaternary ammonium compound a4) is cetyl trimethyl ammonium chloride. The proportion by weight of the quaternary ammonium compound a4), in particular of the cetyl trimethyl ammonium chloride, in the total weight of the cosmetic preparation a) is preferably 0.05 to 2.0% by weight, preferably 0.1 to 1.5% by weight, and in particular 0.2 to 1.0% by weight.

In a further preferred embodiment, the cosmetic preparation a) also includes a non-ionic surfactant a5), preferably a non-ionic surfactant from the group of PEG derivatives of hydrogenated castor oil, particularly preferably PEG-40 Hydrogenated Castor Oil. Particularly preferred non-ionic surfactants are PEG derivatives of hydrogenated castor oil, which are obtainable for example under the name PEG Hydrogenated Castor Oil, such as PEG-30 Hydrogenated Castor Oil, PEG-33 Hydrogenated Castor Oil, PEG-35 Hydrogenated Castor Oil, PEG-36 Hydrogenated Castor Oil, or PEG-40 Hydrogenated Castor Oil. The use of PEG-40 Hydrogenated Castor Oil is particularly preferred in accordance with the invention.

The proportion by weight of the non-ionic surfactant, in particular of PEG-40 Hydrogenated Castor Oil, in the total weight of the cosmetic preparation a) is preferably 0.05 to 2.0% by weight, preferably 0.1 to 1.5% by weight, and in particular 0.2 to 1.0% by weight.

The cosmetic preparation includes at least one film-forming polymer a6) as further preferred ingredient.

On account of their cosmetic effect, film-forming polymers that are used with preference are in particular the polyvinylpyrrolidones (INCI name: PVP) and the vinylpyrrolidone/vinyl acetate copolymers (INCI name VP/VA copolymer), wherein the proportion by weight of these polymers is preferably limited to amounts between 1.0 and 10% by weight. Particularly preferred cosmetic preparations a) therefore contain at least one film-forming polymer from the group of polyvinylpyrrolidone and vinylpyrrolidone/vinyl acetate copolymers.

The proportion by weight of the film-forming polymers a6), which are not identical to the previously described aminofunctional silicones a2), is preferably 0.1 to 10% by weight, preferably 0.5 to 8.0% by weight, and in particular 1.0 to 6.0% by weight, in each case in relation to the total weight of the cosmetic preparation a).

Protein hydrolyzates and/or derivatives thereof can be used as nourishing ingredient. Protein hydrolyzates are product mixtures which are obtained by acid-catalyzed, base-catalyzed or enzymatically catalyzed degradation of proteins. The term “protein hydrolyzates” is understood in accordance with the invention to also mean total hydrolyzates and also individual amino acids and derivatives thereof as well as mixtures of different amino acids. The molecular weight of the protein hydrolyzates usable in accordance with the invention lies between 75, the molecular weight for glycine, and 200,000 daltons, and the molecular weight is particularly preferably 75 to 50,000 daltons, and very particularly preferably 75 to 20,000 daltons.

A further group of nourishing ingredients is constituted by the vitamins, provitamins, vitamin precursors and/or derivatives thereof. Here, vitamins, provitamins and vitamin precursors which are usually assigned to the groups A, B, C, E, F and H are preferred in accordance with the invention.

Further nourishing ingredients are glycerol, propylene glycol, panthenol, caffeine, nicotinamide, and sorbitol.

Plant extract, but also mono- or oligosaccharides and/or lipids can be used as nourishing ingredient.

The cosmetic agents according to the invention are particularly suitable for application by means of a spraying device, wherein the used spraying device can be a pump spray or an aerosol spray. Corresponding cosmetic products therefore comprise, in addition to the above-described cosmetic agents a), also a dispensing device with a spray valve b).

The use of aerosol sprays in which the cosmetic agent a) is sprayed by means of a pressurized propellant gas is preferred. Corresponding cosmetic products comprise, in addition to the above-described cosmetic agent a) and a dispensing device with spray valve b), also a propellant c).

Propellants (propellant gases) which are suitable in accordance with the invention are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, specifically both individually and in combination. Hydrophilic propellant gases, such as carbon dioxide, can also be used advantageously in the sense of the present invention if the proportion of hydrophilic gases is selected to be low and lipophilic propellant gas (for example propane/butane) is present in excess. Propane, n-butane, mixtures of these propellant gases and also dimethylether are particularly preferred. It has been found that the use of propane/butane mixtures provides advantages in terms of production and application.

The ratio by weight of the cosmetic preparation a) to the propellant c) is preferably 20:1 to 1:2, preferably 16:1 to 1:1 and in particular 12:1 to 4:1. Preferred cosmetic agents therefore comprise
a cosmetic preparation a), including, in relation to the total weight of the cosmetic preparation,
a1) 0.01 to 1.0 wt.-% of at least one hydrophobically modified metal oxide powder;
a2) 0.01 to 0.5 wt.-% of at least one aminofunctional silicone;
a3) 40 to 95 wt.-% of water.
a propellant c), preferably dimethylether,
   wherein the ratio by weight of the cosmetic preparation a) to the propellant c) is 20:1 to 1:2, preferably 16:1 to 1:1, and in particular 12:1 to 4:1.

In the case of the aerosol spray, the dispensing device with spray valve usually also comprises a pressure-resistant container. Vessels made of metal (aluminum, tinplate, tin), protected plastic or non-splintering plastic, or made of glass which is coated externally with plastic can be used as pressure-resistant containers, wherein the compressive strength and breaking strength, resistance to corrosion, ease of filling, etc. all play a role when selecting such a vessel. Special inner protective coatings ensure the resistance to corrosion in respect of the cosmetic agent a).

In a preferred embodiment of the invention the valve has a valve stem coated by a lacquer of a polymeric plastic A and an equally flexible element with recovery characteristic, which returns the valve into the closed position (=rest position of the valve) once the actuation is complete. Corresponding cosmetic products in which the aerosol dispensing device comprises a valve that has a valve stem and/or a flexible element with recovery characteristic which are/is coated by a lacquer or a polymeric plastic A are preferred in accordance with the invention.

In a further preferred embodiment of the invention the valve has a flexible element with recovery characteristic and/or a valve stem made of at least one plastic B, preferably made of an elastomeric plastic. Here as well, cosmetic products according to the invention in which the valve comprises a flexible element with recovery characteristic and/or a valve stem made of at least one plastic B are preferred, wherein preferred plastics B are elastomeric plastics. Particularly preferred elastomeric plastics are selected from Buna, in particular Buna N, Buna 421, Buna 1602 and Buna KA 6712, neoprene, butyl and chlorobutyl.

In a further preferred embodiment of the invention the flexible element with recovery characteristic can be formed as a coil spring or coil compression spring. In a further preferred embodiment of the invention the flexible element with recovery characteristic can be formed in one piece with the valve stem and can have flexible legs.

The composition of some preferred cosmetic preparations can be deduced from the following tables (values in wt.-% are based on the total weight of the cosmetic preparation unless specified otherwise).

| | Formula 1 | | Formula 2 | | Formula 3 | | Formula 4 | | Formula 5 | |
|---|---|---|---|---|---|---|---|---|---|---|
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 6 | | Formula 7 | | Formula 8 | | Formula 9 | | Formula 10 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 11 | | Formula 12 | | Formula 13 | | Formula 14 | | Formula 15 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 16 | | Formula 17 | | Formula 18 | | Formula 19 | | Formula 20 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 21 | | Formula 22 | | Formula 23 | | Formula 24 | | Formula 25 | |
| silica dimethyl | 0.01 | to 1.0 | 0.02 | to 1.0 | 0.02 | to 1.0 | 0.03 | to 0.8 | 0.05 | to 0.5 |
| silylate a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 26 | | Formula 27 | | Formula 28 | | Formula 29 | | Formula 30 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| trimethylalkyl- | 0.05 | to 2.0 | 0.1 | to 1.5 | 0.1 | to 1.5 | 0.2 | to 1.0 | 0.2 | to 1.0 |
| ammonium- | | | | | | | | | | |
| halide a4) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 31 | | Formula 32 | | Formula 33 | | Formula 34 | | Formula 35 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| trimethylalkyl- | 0.05 | to 2.0 | 0.1 | to 1.5 | 0.1 | to 1.5 | 0.2 | to 1.0 | 0.2 | to 1.0 |
| ammonium | | | | | | | | | | |
| halide a4) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 36 | | Formula 37 | | Formula 38 | | Formula 39 | | Formula 40 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| trimethylalkyl- | 0.05 | to 2.0 | 0.1 | to 1.5 | 0.1 | to 1.5 | 0.2 | to 1.0 | 0.2 | to 1.0 |
| ammonium | | | | | | | | | | |
| halide a4) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 41 | | Formula 42 | | Formula 43 | | Formula 44 | | Formula 45 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| cetyltrimethyl- | 0.05 | to 2.0 | 0.1 | to 1.5 | 0.1 | to 1.5 | 0.2 | to 1.0 | 0.2 | to 1.0 |
| ammonium | | | | | | | | | | |
| chloride a4) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 46 | | Formula 47 | | Formula 48 | | Formula 49 | | Formula 50 | |
| silica dimethyl | 0.01 | to 1.0 | 0.02 | to 1.0 | 0.02 | to 1.0 | 0.03 | to 0.8 | 0.05 | to 0.5 |
| silylate a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| cetyltrimethyl- | 0.05 | to 2.0 | 0.1 | to 1.5 | 0.1 | to 1.5 | 0.2 | to 1.0 | 0.2 | to 1.0 |
| ammonium | | | | | | | | | | |
| chloride a4) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 51 | | Formula 52 | | Formula 53 | | Formula 54 | | Formula 55 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| non-ionic | 0.05 | to 2.0 | 0.1 | to 1.5 | 0.1 | to 1.5 | 0.2 | to 1.0 | 0.2 | to 1.0 |
| surfactant a5) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 56 | | Formula 57 | | Formula 58 | | Formula 59 | | Formula 60 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| non-ionic | 0.05 | to 2.0 | 0.1 | to 1.5 | 0.1 | to 1.5 | 0.2 | to 1.0 | 0.2 | to 1.0 |
| surfactant a5) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 61 | | Formula 62 | | Formula 63 | | Formula 64 | | Formula 65 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| non-ionic | 0.05 | to 2.0 | 0.1 | to 1.5 | 0.1 | to 1.5 | 0.2 | to 1.0 | 0.2 | to 1.0 |
| surfactant a5) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 66 | | Formula 67 | | Formula 68 | | Formula 69 | | Formula 70 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| PEG-40 | 0.05 | to 2.0 | 0.1 | to 1.5 | 0.1 | to 1.5 | 0.2 | to 1.0 | 0.2 | to 1.0 |
| Hydrogenated | | | | | | | | | | |
| Castor Oil | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 71 | | Formula 72 | | Formula 73 | | Formula 74 | | Formula 75 | |
| silica dimethyl | 0.01 | to 1.0 | 0.02 | to 1.0 | 0.02 | to 1.0 | 0.03 | to 0.8 | 0.05 | to 0.5 |
| silylate a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| PEG-40 | 0.05 | to 2.0 | 0.1 | to 1.5 | 0.1 | to 1.5 | 0.2 | to 1.0 | 0.2 | to 1.0 |
| Hydrogenated | | | | | | | | | | |
| Castor Oil | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 76 | | Formula 77 | | Formula 78 | | Formula 79 | | Formula 80 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| film-forming | 0.05 | to 2.0 | 0.1 | to 1.5 | 0.1 | to 1.5 | 0.2 | to 1.0 | 0.2 | to 1.0 |
| polymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 81 | | Formula 82 | | Formula 83 | | Formula 84 | | Formula 85 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| film-forming | 0.05 | to 2.0 | 0.1 | to 1.5 | 0.1 | to 1.5 | 0.2 | to 1.0 | 0.2 | to 1.0 |
| polymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 86 | | Formula 87 | | Formula 88 | | Formula 89 | | Formula 90 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| film-forming | 0.05 | to 2.0 | 0.1 | to 1.5 | 0.1 | to 1.5 | 0.2 | to 1.0 | 0.2 | to 1.0 |
| polymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula 91 | | Formula 92 | | Formula 93 | | Formula 94 | | Formula 95 | |
| pyrogenic | 0.01 | to 1.0 | 0.02 | to 1.0 | 0.02 | to 1.0 | 0.03 | to 0.8 | 0.05 | to 0.5 |
| silicon dioxide | | | | | | | | | | |
| hydrophobized | | | | | | | | | | |
| by silanization | | | | | | | | | | |
| a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| VP/VA | 0.05 | to 2.0 | 0.1 | to 1.5 | 0.1 | to 1.5 | 0.2 | to 1.0 | 0.2 | to 1.0 |
| copolymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | | | | | | | | | Formula | |
| | Formula 96 | | Formula 97 | | Formula 98 | | Formula 99 | | 100 | |
| silica dimethyl | 0.01 | to 1.0 | 0.02 | to 1.0 | 0.02 | to 1.0 | 0.03 | to 0.8 | 0.05 | to 0.5 |
| silylate a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| VP/VA | 0.05 | to 2.0 | 0.1 | to 1.5 | 0.1 | to 1.5 | 0.2 | to 1.0 | 0.2 | to 1.0 |
| copolymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula | | Formula | | Formula | | Formula | | Formula | |
| | 101 | | 102 | | 103 | | 104 | | 105 | |
| hydrophobically | 0.01 | to 1.0 | 0.02 | to 1.0 | 0.02 | to 1.0 | 0.03 | to 0.8 | 0.05 | to 0.5 |
| modified metal | | | | | | | | | | |
| oxide powder | | | | | | | | | | |
| a1) | | | | | | | | | | |
| aminofunctional | 0.01 | to 0.5 | 0.01 | to 0.5 | 0.02 | to 0.4 | 0.03 | to 0.3 | 0.05 | to 0.2 |
| silicone a2) | | | | | | | | | | |
| trimethylalkyl- | 0.05 | to 2.0 | 0.1 | to 1.5 | 0.1 | to 1.5 | 0.2 | to 1.0 | 0.2 | to 1.0 |
| ammonium | | | | | | | | | | |
| halide a4) | | | | | | | | | | |
| non-ionic | 0.05 | to 2.0 | 0.1 | to 1.5 | 0.1 | to 1.5 | 0.2 | to 1.0 | 0.2 | to 1.0 |
| surfactant a5) | | | | | | | | | | |
| film-forming | 0.05 | to 2.0 | 0.1 | to 1.5 | 0.1 | to 1.5 | 0.2 | to 1.0 | 0.2 | to 1.0 |
| polymer a6) | | | | | | | | | | |
| water | 40 | to 95 | 40 | to 95 | 50 | to 95 | 70 | to 95 | 80 | to 95 |
| optional | to 100 | | to 100 | | to 100 | | to 100 | | to 100 | |
| additives | | | | | | | | | | |
| | Formula | | Formula | | Formula | | Formula | | Formula | |
| | 101 | | 102 | | 103 | | 104 | | 105 | |
| silica dimethyl | 0.01 | to 1.0 | 0.02 | to 1.0 | 0.02 | to 1.0 | 0.03 | to 0.8 | 0.05 | to 0.5 |
| silylate a1) | | | | | | | | | | |
| amodimethicone | 0.01 | to 0.5 | 0.01 | to 0.5 | 0.02 | to 0.4 | 0.03 | to 0.3 | 0.05 | to 0.2 |
| a2) | | | | | | | | | | |
| cetyl trimethyl | 0.05 | to 2.0 | 0.1 | to 1.5 | 0.1 | to 1.5 | 0.2 | to 1.0 | 0.2 | to 1.0 |
| ammonium | | | | | | | | | | |
| chloride a4) | | | | | | | | | | |
| PEG-40 | 0.05 | to 2.0 | 0.1 | to 1.5 | 0.1 | to 1.5 | 0.2 | to 1.0 | 0.2 | to 1.0 |
| Hydrogenated | | | | | | | | | | |
| Castor Oil a5) | | | | | | | | | | |
| VP/VA | 0.05 | to 2.0 | 0.1 | to 1.5 | 0.1 | to 1.5 | 0.2 | to 1.0 | 0.2 | to 1.0 |
| copolymer a6) | | | | | | | | | | |
| water | 40 | to 95 | 40 | to 95 | 50 | to 95 | 70 | to 95 | 80 | to 95 |

| | | | | | |
|---|---|---|---|---|---|
| optional | to 100 | to 100 | to 100 | to 100 | to 100 |
| additives | | | | | |

As mentioned at the outset, the previously described cosmetic agents are characterized by properties that are relevant in particular for hair cosmetics, in particular advantageous properties with regard to temporary hair shaping. A second subject of the present invention is therefore the use of an agent according to the invention for temporarily shaping keratin-containing fibers, in particular human hair.

A third subject of the present invention is a method for temporarily shaping keratin-containing fibers, in particular human hair, in which a cosmetic agent according to the invention is applied to the keratin fibers and the shape of said fibers is temporarily fixed.

The agents, uses and methods according to the invention and some of the preferred embodiments therefore are characterized by the following points:

A cosmetic agent for temporarily shaping keratin fibers, comprising

a) a cosmetic preparation including, based on the total weight of the cosmetic preparation,
a1) 0.01 to 1.0 wt.-% of at least one hydrophobically modified metal oxide powder;
a2) 0.01 to 0.5 wt.-% of at least one aminofunctional silicone;
a3) 40 to 95 wt.-% of water.

The cosmetic agent as described herein, characterized in that the proportion by weight of the hydrophobically modified metal oxide powder a1) in the total weight of the cosmetic preparation a) is 0.02 to 1.0 wt.-%, preferably 0.03 to 0.8 wt.-%, and in particular 0.05 to 0.5 wt.-%.

The cosmetic agent as described herein, characterized in that the hydrophobically modified metal oxide powder a1) is selected from the group of hydrophobized silicates obtained by silanization of pyrogenic silicon dioxide.

The cosmetic agent as described herein, characterized in that the hydrophobically modified metal oxide powder a1) is selected from the compounds with the INCI names Silica Dimethyl Silylate (for example Aerosil R792, Aerosil R794), Silica Dimethicone Silylate (for example Aerosil R202) and Silica Silylate (for example Aerosil R805, Aerosil R812, Aerosil R816), preferably from the compounds with the INCI name Silica Dimethyl Silylate.

The cosmetic agent as described herein, characterized in that the proportion by weight of the aminofunctional silicone a2) in the total weight of the cosmetic preparation a) is 0.02 to 0.4 wt.-%, preferably 0.03 to 0.3 wt.-%, and in particular 0.05 to 0.2 wt.-%.

The cosmetic agent as described herein, characterized in that the aminofunctional silicone a2) is selected from the compounds with the INCI name Amodimethicone.

The cosmetic agent as described herein, characterized in that the aminofunctional silicone a2) is a functionalized aminofunctional silicone, preferably a hydroxylamino-modified silicone.

The cosmetic agent as described herein, characterized in that the cosmetic preparation also comprises a quaternary ammonium compound a4) different from the aminofunctional silicone a2), preferably a quaternary ammonium compound from the group of the trimethylalkylammonium halides.

The cosmetic agent as described herein, characterized in that the cosmetic preparation also comprises a non-ionic surfactant a5), preferably a non-ionic surfactant from the group of the PEG derivatives of hydrogenated castor oil, particularly preferably PEG-40 Hydrogenated Castor Oil.

The cosmetic agent as described herein, characterized in that the cosmetic preparation also comprises a film-forming polymer a6), preferably a film-forming polymer from the group of polyvinylpyrrolidone and vinylpyrrolidone/vinyl acetate copolymers.

The cosmetic agent as described herein, characterized in that the proportion by weight of the water a3) in the total weight of the cosmetic preparation a) is 60 to 95 wt.-%, preferably 70 to 95 wt.-%, and in particular 80 to 95 wt.-%.

A cosmetic product, comprising

a) a cosmetic agent as described herein;
b) a dispensing device with spray valve.

A cosmetic product, comprising

a) a cosmetic agent as described herein;
b) a dispensing device with spray valve;
c) a propellant.

The cosmetic product as described herein, characterized in that the ratio by weight of the cosmetic preparation a) to the propellant c) is 20:1 to 1:2, preferably 16:1 to 1:1 and in particular 12:1 to 4:1.

Use of an agent or a product as described herein for temporarily shaping keratin-containing fibers, in particular human hair.

A method for temporarily shaping keratin-containing fibers, in particular human hair, in which a cosmetic agent as described herein is applied to the keratin fibers and the shape of said fibers is temporarily fixed.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic agent for temporarily shaping keratin fibers, comprising
a) a cosmetic preparation including, based on the total weight of the cosmetic preparation,
a1) 0.01 to 1.0 wt.-% of at least one hydrophobically modified metal oxide powder;
a2) 0.01 to 0.5 wt.-% of at least one aminofunctional silicone;
a3) 40 to 95 wt.-% of water.

2. The cosmetic agent according to claim 1, wherein the proportion by weight of the hydrophobically modified metal oxide powder a1) in the total weight of the cosmetic preparation a) is 0.02 to 1.0 wt.-%.

3. The cosmetic agent according to claim 1, wherein the proportion by weight of the hydrophobically modified metal oxide powder a1) in the total weight of the cosmetic preparation a) is 0.03 to 0.8 wt.-%.

4. The cosmetic agent according to claim 1, wherein the proportion by weight of the hydrophobically modified metal oxide powder a1) in the total weight of the cosmetic preparation a) is 0.05 to 0.5 wt.-%.

5. The cosmetic agent according to claim 1, wherein the hydrophobically modified metal oxide powder a1) at least one hydrophobized silicate obtained by silanization of pyrogenic silicon dioxide.

6. The cosmetic agent according to claim 1, wherein the proportion by weight of the aminofunctional silicone a2) in the total weight of the cosmetic preparation a) is 0.02 to 0.4 wt.-%.

7. The cosmetic agent according to claim 1, wherein the proportion by weight of the aminofunctional silicone a2) in the total weight of the cosmetic preparation a) is 0.03 to 0.3 wt.-%.

8. The cosmetic agent according to claim 1, wherein the proportion by weight of the aminofunctional silicone a2) in the total weight of the cosmetic preparation a) is 0.05 to 0.2 wt.-%.

9. The cosmetic agent according to claim 1, wherein the aminofunctional silicone a2) is selected from the compounds with the INCI name Amodimethicone.

10. The cosmetic agent according to claim 1, wherein the proportion by weight of the water a3) in the total weight of the cosmetic preparation a) is 60 to 95 wt.-%.

11. The cosmetic agent according to claim 1, wherein the proportion by weight of the water a3) in the total weight of the cosmetic preparation a) is 70 to 95 wt.-%.

12. The cosmetic agent according to claim 1, wherein the proportion by weight of the water a3) in the total weight of the cosmetic preparation a) is 80 to 95 wt.-%.

13. A cosmetic product, comprising
a) a cosmetic agent according to claim 1;
b) a dispensing device with spray valve.

14. A cosmetic product, comprising
a) a cosmetic agent according claim 1;
b) a dispensing device with spray valve;
c) a propellant.

15. A method for temporarily shaping keratin-containing fibers, comprising applying to keratin fibers and shaping the keratin fibers.
